(19) United States
(12) Patent Application Publication
(10) Pub. No.: US 2009/0098059 A1
(43) Pub. Date: Apr. 16, 2009

(54) CONTRAST AGENTS

(76) Inventor: Duncan Wynn, Amersham (GB)

Correspondence Address: GE HEALTHCARE, INC., IP DEPARTMENT, 101 CARNEGIE CENTER, PRINCETON, NJ, 08540-6231, US

(21) Appl. No.: 12/299,793

(22) PCT Filed: May 11, 2007
(86) PCT No.: PCT/NO07/00171

§ 371 Date: Nov. 6, 2008

(30) Foreign Application Priority Data
May 11, 2006 (NO) 20062122

(51) Int. Cl. A61K 49/04; C07C 237/46; C07C 229/40; A61K 49/00

(52) U.S. Cl. 424/9.452; 564/153; 560/129; 424/9.1

(57) ABSTRACT

The present invention relates to iodine containing compounds containing a central optionally substituted cyclohexane central moiety allowing for the arrangement of three iodinated phenyl groups bound thereto. The invention also relates to the use of such diagnostic compositions as contrast agents in diagnostic imaging and in particular in X-ray imaging and to contrast media containing such compounds.

### TECHNICAL FIELD OF THE INVENTION

The present invention relates to a class of compounds and to diagnostic compositions containing such compounds where the compounds are iodine containing compounds. More specifically the iodine containing compounds are chemical compounds containing an optionally substituted cyclohexane central moiety allowing for the arrangement of three iodinated phenyl groups bound thereto.

The invention also relates to the use of such diagnostic compositions as contrast agents in diagnostic imaging and in particular in X-ray imaging and to contrast media containing such compounds.

### DESCRIPTION OF RELATED ART

All diagnostic imaging is based on the achievement of different signal levels from different structures within the body. Thus in X-ray imaging for example, for a given body structure to be visible in the image, the X-ray attenuation by that structure must differ from that of the surrounding tissues. The difference in signal between the body structure and its surroundings is frequently termed contrast and much effort has been devoted to means of enhancing contrast in diagnostic imaging since the greater the contrast between a body structure and its surroundings the higher the quality of the images and the greater their value to the physician performing the diagnosis. Moreover, the greater the contrast the smaller the body structures that may be visualized in the imaging procedures, i.e. increased contrast can lead to increased spatial resolution.

The diagnostic quality of images is strongly dependent on the inherent noise level in the imaging procedure, and the ratio of the contrast level to the noise level can thus be seen to represent an effective diagnostic quality factor for diagnostic images.

Achieving improvement in such a diagnostic quality factor has long been and still remains an important goal. In techniques such as X-ray, magnetic resonance imaging (MRI) and ultrasound, one approach to improving the diagnostic quality factor has been to introduce contrast enhancing materials formulated as contrast media into the body region being imaged.

Thus in X-ray early examples of contrast agents were insoluble inorganic barium salts which enhanced X-ray attenuation in the body zones into which they distributed. For the last 50 years the field of X-ray contrast agents has been dominated by soluble iodine containing compounds. Commercial available contrast media containing iodinated contrast agents are usually classified as ionic monomers such as diatrizoate (marketed e.g. under the trade name Gastrografen™), ionic dimers such as ioxaglate (marketed e.g. under the trade name Hexabrix™), nonionic monomers such as iohexyl (marketed e.g. under the trade name Omnipaque™), iopamidol (marketed e.g. under the trade name Isovue™), iomeprol (marketed e.g. under the trade name Iomeron™) and the non-ionic dimer iodixanol (marketed under the trade name and Visipaque™).

The most widely used commercial non-ionic X-ray contrast agents such as those mentioned above are considered safe. Contrast media containing iodinated contrast agents are used in more than 20 millions of X-ray examinations annually in the USA and the number of adverse reactions is considered acceptable. However, since a contrast enhanced X-ray examination will require up to about 200 ml contrast media administered in a total dose, there is a continuous drive to provide improved contrast media.

The utility of the contrast media is governed largely by its toxicity, by its diagnostic efficacy, by adverse effects it may have on the subject to which the contrast medium is administered and by the ease of storage and ease of administration. Since such media are conventionally used for diagnostic purposes rather than to achieve direct therapeutic effect, it is generally desirable to provide media having as little as possible effect on the various biological mechanisms of the cells or the body as this will lead to lower toxicity and lower adverse clinical effect. The toxicity and adverse biological effects of a contrast medium are contributed to by the components of the formulation medium, e.g. the solvent or carrier as well as the contrast agent itself and its components such as ions for the ionic contrast agents and also by its metabolites.

The major contributing factors to the toxicity of the contrast medium are identified as the chemotoxicity of the contrast agent, the osmolality of the contrast medium and the ionic composition or lack thereof of the contrast medium.

Desirable characteristics of an iodinated contrast agent are low toxicity of the compound itself (chemotoxicity), low viscosity of the contrast medium wherein the compound is dissolved, low osmolality of the contrast medium and a high iodine content (frequently measured in g iodine per ml of the formulated contrast medium for administration). The iodinated contrast agent must also be completely soluble in the formulation medium, usually an aqueous medium and remain in solution during storage.

The osmolality of the commercial products, and in particular of the non-ionic compounds, is acceptable for most media containing dimers and non-ionic monomers although there is still room for improvement. In coronary angiography for example, injection into the circulatory system of a bolus dose of contrast medium has caused severe side effects. In this procedure contrast medium rather than blood flows through the system for a short period of time, and differences in the chemical and physiochemical nature of the contrast medium and the blood that it replaces can cause undesirable adverse effects such as arrhythmias, QT prolongation and reduction in cardiac contractive force. Such effects are seen in particular with ionic contrast agents where osmotoxic effects are associated with hypertonicity of the injected contrast medium. Contrast media that are isotonic or slightly hypotonic with the body fluids are particularly desired. Low osmolar contrast media have low renal toxicity which is particularly desirable. The osmolality is a function of the number of particles per volume unit of the formulated contrast medium.

To keep the injection volume of the contrast media as low as possible it is highly desirable to formulate contrast media with high concentration of iodine/ml, and still maintain the osmolality of the media at a low level, preferably below or close to isotonicity. The development of non-ionic monomeric contrast agents and in particular non-ionic bis(triiodophenyl) dimers such as iodixanol (EP patent 108638) has provided contrast media with reduced osmotoxicity allowing contrast effective iodine concentration to be achieved with hypotonic solution, and even allowed correction of ionic imbalance by inclusion of plasma ions while still maintaining the contrast medium Visipaque™ at the desired osmolality (WO 90/01194 and WO 91/13636).

X-ray contrast media at commercial high iodine concentration have relative high viscosity, ranging from about 15 to 60 mPas at ambient temperature. Generally, contrast media where the contrast enhancing agent is a dimer has higher viscosity than the corresponding contrast media where the contrast enhancing agent is the monomer corresponding to the dimer. Such high viscosities may pose problems to the administrators of the contrast medium, requiring relatively large bore needles or high applied pressure, and are particularly pronounced in pediatric radiography and in radiographic techniques which require rapid bolus administration, e.g. in angiography.

X-ray contrast agents of high molecular weight has been proposed, e.g. polymers with substituted triiodinated phenyl groups grafted on the polymer, see EP 354836, EP 436316 and U.S. Pat. No. 5,019,370. Further, WO 9501966, EP 782563 and U.S. Pat. No. 5,817,873 read on compounds having e.g. 3 and 4 substituted triiodinated phenyl groups arranged linearly or around a central core. However, none of these proposed compounds are on the market.

Hence there still exists a desire to develop contrast agents that solves one or more of the problems discussed above. Such agents should ideally have improved properties over the soluble iodine containing compounds in one or more of the following properties: renal toxicity, osmolality, viscosity, solubility, injection volumes/iodine concentration and attenuation/radiation dose.

### SUMMARY OF THE INVENTION

The present invention provides compounds useful as contrast media having improved properties over the known media with regards to at least one of the following criteria osmolality (and hence the renal toxicity), viscosity, iodine concentration and solubility. The contrast media comprises iodine containing contrast enhancing compounds where iodine containing compounds are chemical compounds containing a optionally substituted cyclohexane central moiety allowing for the arrangement of three iodinated phenyl groups bound to thereto. The iodine containing contrast enhancing compounds can be synthesized from commercially available and relatively inexpensive starting materials.

### DETAILED DESCRIPTION OF THE INVENTION

The contrast media comprises iodine containing contrast enhancing compounds of formula (I)

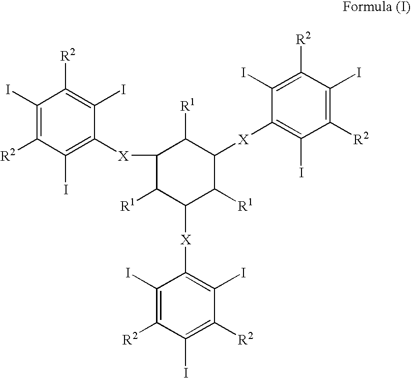

wherein each R¹ are the same and different and denotes a hydrogen atom or a hydroxyl group;
each R² are the same or different and denote a hydrogen atom or a non-ionic hydrophilic moiety, provided that at least one of the R² groups represent a non-ionic hydrophilic moiety;
each X are the same or different and denote a bridging group of the formulas
*—(CH₂)ₙ—NR—CO— and *—CO—NR—(CH₂)ₙ— where

R denotes a hydrogen atom or an acyl moiety;

n denotes an integer of 0 to 4; and

* denotes the binding to the central cyclohexane moiety

and salts or optical active isomers thereof.

The central cyclohexane moiety exerts certain constraints on the structure of the compound of formula (I) e.g. by locking the iodinate phenyl groups in specific positions relative to each other and to the central cyclohexane moiety. Hence, the diameter and the molecular volume of the compound of formula (I) will be relatively small and the compound will assume a relatively compact 3-dimensional configuration. Further, if the compounds can exist in isomeric forms, they should preferably be locked into one isomeric form and preferably the isomeric form with the smallest diameter, provided that the solubility of this isomer is satisfactory. The hydrophilic R² groups present their hydrophilic groups at the surface of the molecule of formula (I) contributing to the hydrophilic properties of the compound.

The hydrophilic moieties R² may be any of the non-ionizing groups conventionally used to enhance water solubility. Suitable groups include straight chain or branched chain C₁₋₁₀ alkyl groups, preferably C₁₋₅ alkyl groups, optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms. Particular preferred examples include polyhydroxyalkyl, hydroxyalkoxyalkyl and hydroxypolyalkoxyalkyl and such groups attached to the phenyl group via an amide linkage such as hydroxyalkylaminocarbonyl, N-alkyl-hydroxyalkylaminocarbonyl and bis-hydroxyalkylaminocarbonyl groups.

In a preferred embodiment the hydrophilic moieties R² are selected from the groups listed below and are preferably containing 1 to 6 hydroxy groups, more preferably 1 to 3 hydroxy groups. Examples of preferred groups comprise groups of the formulas:

—CONH—CH₂—CH₂OH

—CONH—CH₂—CHOH—CH₂OH

—CON(CH₃)CH₂—CHOH—CH₂OH

—NHCOCH₂OH

—N(COCH₃)H

—N(COCH₃)C₁₋₃ alkyl

—N(COCH₃)— mono, bis or tris-hydroxy C₁₋₄ alkyl

—N(COCH₂OH)— hydrogen, mono, bis or tris-hydroxy C₁₋₄ alkyl

—N(CO—CHOH—CH₂OH)— hydrogen, mono, bis or trihydroxylated C₁₋₄ alkyl.

N(CO—CHOH—CHOH—CH₂OH)— hydrogen, mono, bis or trihydroxylated C₁₋₄ alkyl.

—CON(CH₂—CHOH—CH₂OH)(CH₂—CH₂OH)

—CONH—C(CH₂OH)₃ and

—CONH—CH(CH₂OH)(CHOH—CH₂OH).

More preferably the R² groups will be equal or different and denote one or more moieties of the formulas —CON(CH₃)CH₂—CHOH—CH₂OH, —CONH—CH₂—CHOH—CH₂OH, —CONH—CH—(CH₂OH)₂, —CON—(CH₂—CH₂OH)₂, —CON—(CH₂—CHOH—CH₂OH)₂, —NHCOCH₂OH and —N(COCH₂OH)— mono, bis or tris-hydroxy C₁₋₄ alkyl.

All the R² groups may also be equal and denote one of the preferred moieties and most preferred the moiety —CONH—CH₂—CHOH—CH₂OH.

The R¹ groups preferably all denote hydrogen or all denote hydroxyl. Most preferably all the R¹ groups denote hydrogen.

The bridging groups X of formulas *—(CH₂)ₙ—NR—CO— and *—CO—NR—(CH₂)ₙ— are bond to the cyclohexane ring structure with the atom marked with the asterisk *. The opposite end of the bridging group is then attached to the triiodinated phenyl group. When n denotes 0, the binding to the cyclohexyl or the phenyl group will be through the nitrogen atom, when n is 1, 2, 3 or 4 the binding will be through a carbon atom.

Each R preferably denotes hydrogen atom and residues of aliphatic organic acids, and in particular a C₁ to C₅ organic acid such as formyl, acetyl, propionyl, butyryl, isobutyryl and valeryl moieties. Hydroxylated acyl moieties are also feasible. In a further preferred embodiment all groups R are the same. In a particularly preferred embodiment all R groups in the compound of formula (I) are the same and denote the acetyl moieties or are hydrogen atoms.

Particularly preferred bridging groups X are or the formulas *—CH₂—NH—CO—, *—NH—CO— and *—CO—NH—.

In a particularly preferred embodiment the compound of formulas (IIa) to (IIc) are provided:

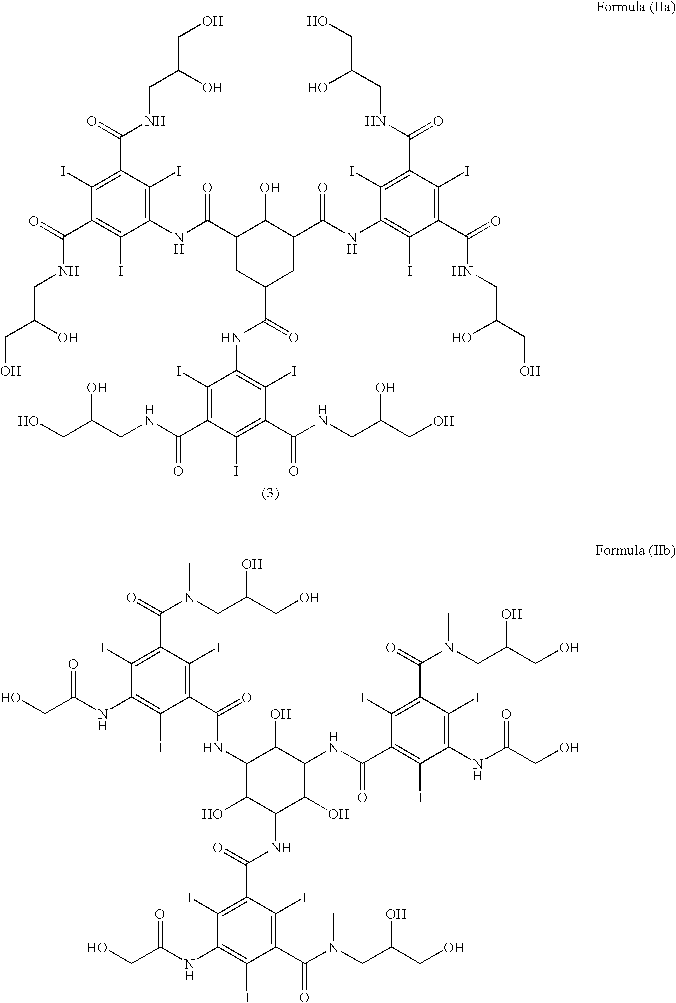

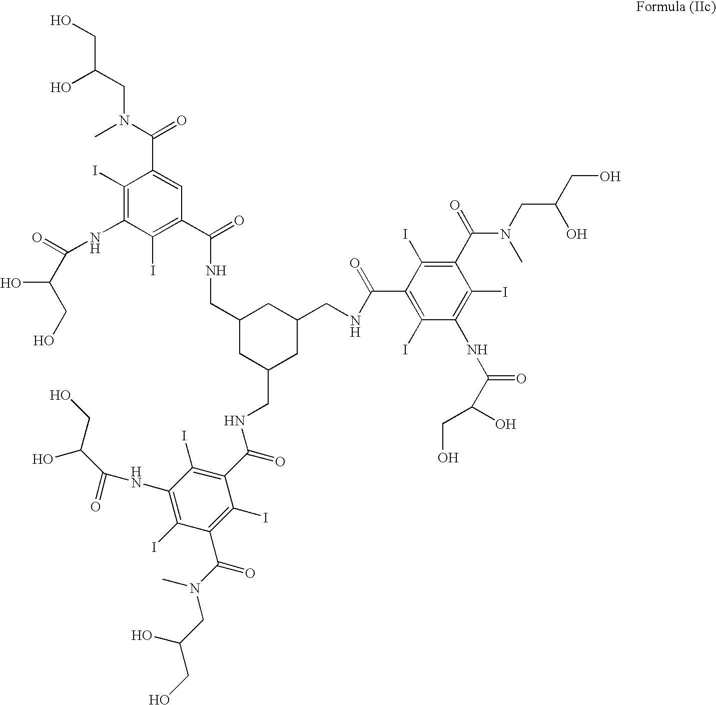

At an iodine concentration of 320 mg/ml, which is a common concentration for commercially available iodinated contrast media, the concentration of the compound of formula (I) will be approximately 0.28 M (Molar). The contrast medium will also be hypoosmolar at this iodine concentration, and this is an advantageous property with regards to the nephrotoxicity of the contrast medium. It is also possible to add electrolytes to the contrast medium to lower the cardiovascular effects as explained in WO 90/01194 and WO 91/13636.

Compounds of formula (I) also comprises stereoisomers and optical active isomers. Both enantiomerically pure products as well as mixtures of optical isomers are included.

The compounds of the invention may be used as contrast agents and may be formulated with conventional carriers and excipients to produce diagnostic contrast media.

Thus viewed from a further aspect the invention provides a diagnostic composition comprising a compound of formula (I) as described above together with at least one physiologically tolerable carrier or excipient, e.g. in aqueous solution for injection optionally together with added plasma ions or dissolved oxygen.

The contrast agent composition of the invention may be in a ready to use concentration or may be a concentrate form for dilution prior to administration. Generally compositions in a ready to use form will have iodine concentrations of at least 100 mg l/ml, preferably at least 150 mg l/ml, with concentrations of at least 300 mg l/ml, e.g. 320 mg l/ml being preferred. The higher the iodine concentration, the higher is the diagnostic value in the form of X-ray attenuation of the contrast media. However, the higher the iodine concentration the higher is the viscosity and the osmolality of the composition. Normally the maximum iodine concentration for a given contrast media will be determined by the solubility of the contrast enhancing agent, e.g. the iodinated compound, and the tolerable limits for viscosity and osmolality.

For contrast media which are administered by injection or infusion, the desired upper limit for the solution's viscosity at ambient temperature (20° C.) is about 30 mPas, however viscosities of up to 50 mPas and even up to 60 mPas can be tolerated. For contrast media given by bolus injection, e.g. in angiographic procedures, osmotoxic effects must be considered and preferably the osmolality should be below 1 Osm/kg H₂O, preferably below 850 mOsm/kg H₂O and more preferably about 300 mOsm/kg H₂O.

With the compounds of the invention such viscosity, osmolality and iodine concentrations targets can be met. Indeed effective iodine concentrations will be reached with hypotonic solutions. It may thus be desirable to make up the solution's tonicity by the addition of plasma cations so as to reduce the toxicity contribution that derives from the imbalance effects following bolus injection. Such cations will desirably be included in the ranges suggested in WO 90/01194 and WO 91/13636.

In particular, addition of sodium and calcium ions to provide a contrast medium that is isotonic with blood for all iodine concentrations, is desirable and obtainable. The plasma cations may be provided in the form of salts with physiologically tolerable counterions, e.g. chloride, sulphate, phosphate, hydrogen carbonate etc., with plasma anions preferably being used.

The compounds of the general formula (I) can be synthesized from available starting materials by several synthetic pathways known or obvious to the skilled artisan. Hence, the compounds of formula (I) are prepared by reacting a derivative of the optionally substituted cyclohexane central group with a reactive derivative of the triiodinated phenyl group.

Tri-iodinated phenyl groups are commercially available or can be produced following procedures described or referred to e.g. in WO95/35122 and WO98/52911. The preferred tri-iodinated compound 5-amino-2,4,6-triiodo-N,N′-bis(2,3-dihydroxypropyl)-isophtalamide is commercially available e.g. from Fuji Chemical Industries, Ltd. The corresponding 5-N-acylated compound can be produced by acetylation with acetic acid anhydride, e.g. as described in U.S. Pat. No. 4,250,113.

The manufacture of triodinated R² substituted anilines are illustrated for example in EP 0108638 where a compound denoted compound A has a preferred structure. When the substituted aniline comprises reactive functions such as hydroxyl groups, these functions should be protected, e.g. by acetylation and the protecting groups will be removed in an additional step.

In one embodiment, cyclohexane-cis,cis-1,3,5-tricarbonyl trichloride is prepared from cis,cis-1,3,5-cyclohexanetricarboxylic acid by standard methods. The acid chloride is treated with an excess of a triiodinated R² substituted aniline.

Hence, when the cyclohexane moiety binds the iodinated phenyl groups by linkers X of the formula *—CO—NR—(CH₂)ₙ—, then a 1,3,5-cyclohexanetricarboxylic acid is reacted with a triiodophenyl group having a free amino group as illustrated below:

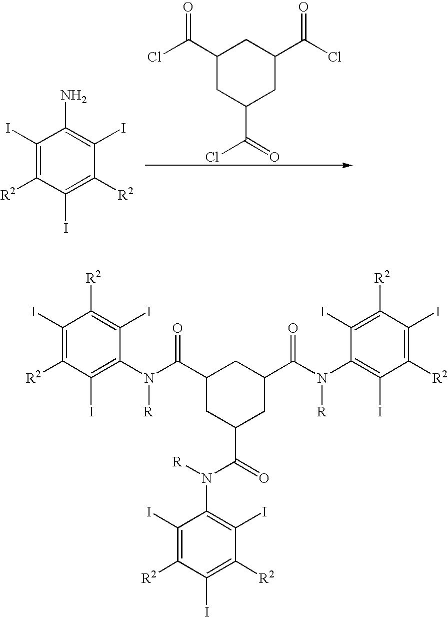

When the cyclohexane moiety binds the iodinated phenyl groups by linkers X of the formula *—(CH₂)ₙ—NR—CO—, then a 1,3,5-cyclohexanetriamine or derivative thereof can be reacted with a triiodophenyl group having a acidic function as illustrated for the bridging group *—CH₂—NH—CO— below.

1,3,5-tris(aminomethyl)cyclohexane is available in 4 steps from the commercially available triester and is purified by preparative HPLC on a small scale or by crystallization on a large scale.

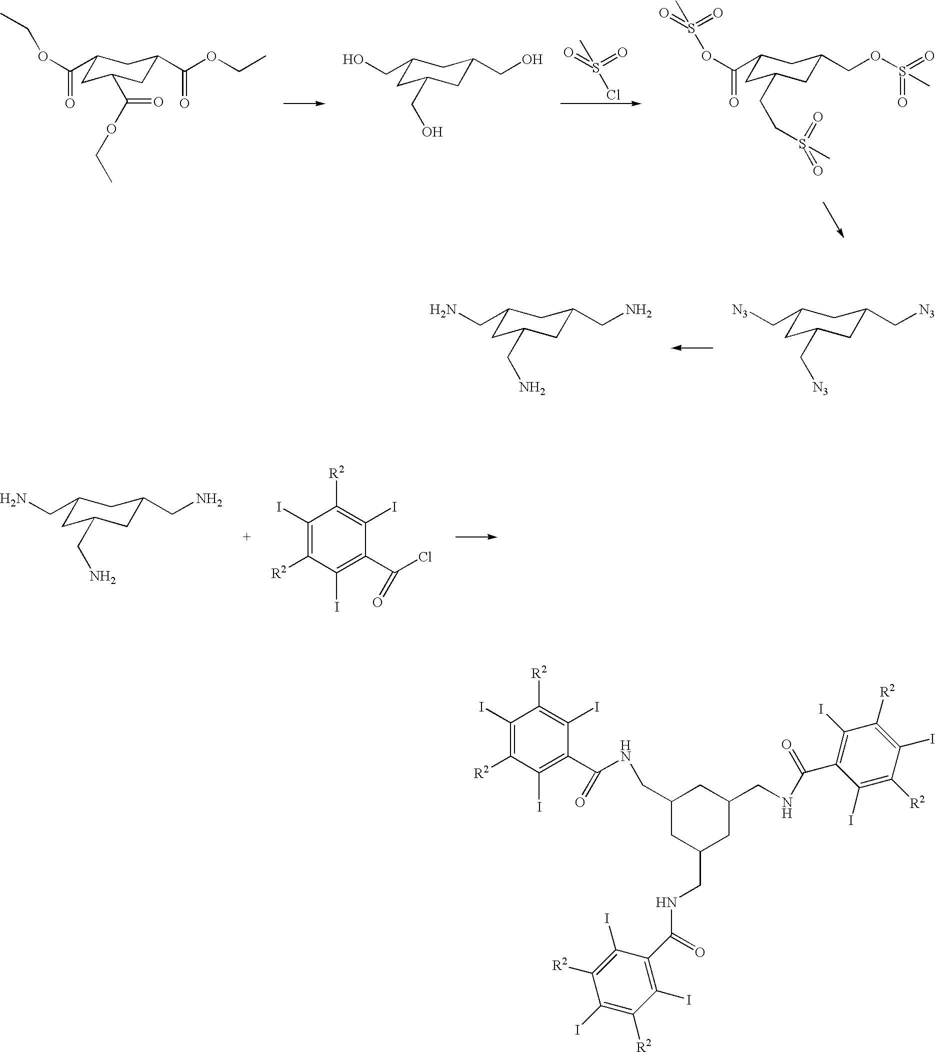

Compounds of formula (I) where the R¹ groups denote hydroxy) groups can be prepared following the procedures above using the corresponding cyclohexane-1,3,5-triols. The triol intermediates can be synthesised from phloroglucinol which is treated with acetyl chloride or acetic anhydride to form phloroglucinol triacetate. Phloroglucinol triacetate is nitrated using fuming nitric acid to give the trinitrophloroglucinol. Trinitrophloroglucinol is then reduced using PtO₂ and hydrogen in aqueous sulphuric acid to give 2,4,6-triamino-cyclohexane-1,3,5-triol.

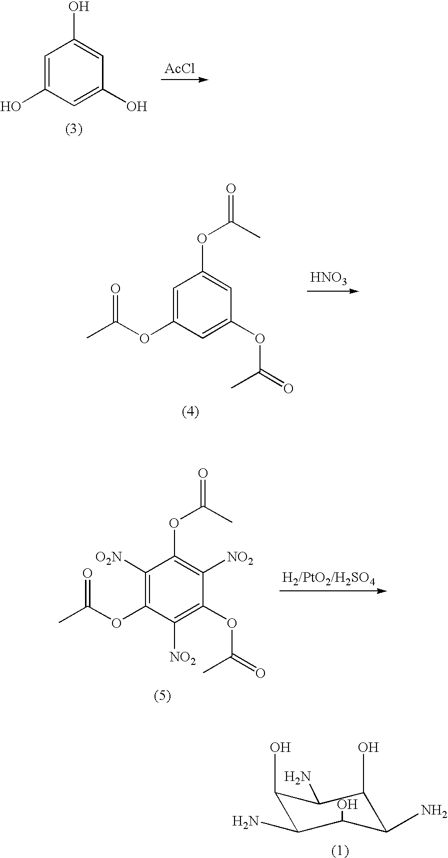

The synthesis of 2,4,6-Triamino-cyclohexane-1,3,5-triol is also described in Helv. Chim. Acta.; 1990; 73; 97 and references therein and also illustrated above.

Compounds of formula (I) are prepared as illustrated above using the 2,4,6-triamino-cyclohexane-1,3,5-triol (1) as starting material as illustrated in Example 5 below.

### EXAMPLES

The preparation of compounds of formula (I) will now be illustrated by the following non-limiting example:

### Example 1

### N¹,N³,N⁵-tris(3,5-bis(2,3-dihydroxypropylcarbamoyl)-2,4,6-triiodophenyl)cyclohexane-1,3,5-tricarboxamide

### a) 5-Amino-2,4,6-triiodo-isophthaloyl dichloride

5-Amino-2,4,6-triiodo-isophtalic acid (30 g, 0.054 mol), thionyl chloride (8.2 ml, 0.113 mol) and pyridine (0.2 ml) in 1,2 dichloroethane (20 ml) were heated to 70° C. A portion of thionyl chloride (15.2 ml, 0.21 mol) was added dropwise during 1½ to 2 hrs, and the mixture was heated to 85° C. for 6 hrs. After cooling the reaction mixture to room temperature, it was poured into 300 g of ice-water. The yellow precipitate that formed was filtered off, sucked dry and then washed with water until washings showed a pH of ca 5. The filter cake was then dried in a vacuum oven at 50° C. for 3 hrs. A light yellow powder was obtained 31 g (˜quant.) as the desired product.

¹³C NMR (DMSOd₆) 66, 78.4, 148.9, 149.2, 169

MS (ES−) found 593.5 [M-H+], expected 593.7

### b) N,N′-Diallyl-5-amino-2,4,6-triiodo-isophthalamide

To a solution of 5-Amino-2,4,6-triiodo-isophthaloyl dichloride in dichloromethane was added allylamine (4 equivalents) under a nitrogen atmosphere at ambient temperature. The reaction was stirred for 18 hours. This yielded a precipitate which was found to be the desired product. ¹H NMR (DMSOd₆) 8.62 (2H, t, j 4.5 Hz), 5.90 (2H, m), 5.46 (2H, br s), 5.37 (2H, d, j 9 Hz), 5.14 (2H, d, 6 Hz), 3.84 (4H, t, 3 Hz). ¹³C (DMSOd₆) 169.6, 149.0, 147.4, 116.0, 79.7, 41.5.

### c) Cyclohexane-cis,cis-1,3,5-tricarbonyl trichloride

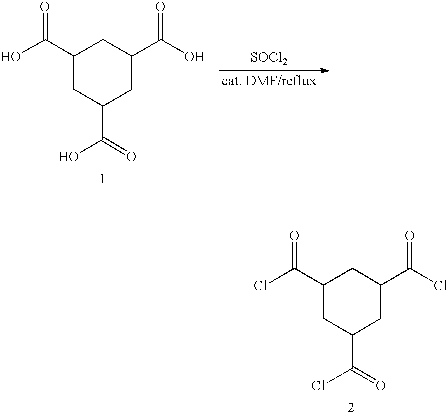

To a suspension of cis,cis-1,3,5-cyclohexanetricarboxylic acid (500 mg, 2.30 mmol) in thionyl chloride (8 ml) was added catalytic amount of DMF and the mixture heated at reflux for 2 hours. After cooling to ambient temperature, excess thionyl chloride was thoroughly evaporated to furnish yellow syrup which solidified on standing in the refrigerator. The crude product was used without further purification.

Yield: 658 mg

### d) Cyclohexane-1,3,5-tricarboxylic acid tris-[(3,5-bis-allylcarbamoyl-2,4,6-triiodo-phenyl)-amide]

To a solution of N,N′-Diallyl-5-amino-2,4,6-triiodo-isophthalamide in N,N-dimethylacetamide at 0° C. will be added dropwise a solution of cyclohexane-1,3,5-tricarbonyl trichloride (1 equivalent) and triethylamine (3.3 equivalent). The mixture will be allowed to warm to room temperature. The mixture will be purified by silica gel chromatography.

### e) N¹,N³,N⁵-tris(3,5-bis(2,3-dihydroxypropylcarbamoyl)-2,4,6-triiodophenyl)cyclohexane-1,3,5-tricarboxamide

To a solution of cyclohexane-1,3,5-tricarboxylic acid tris-[(3,5-bis-allylcarbamoyl-2,4,6-triiodo-phenyl)-amide] dissolved in a mixture of acetone/water (9/1) will be added a solution of osmium catalyst (OsO₄, t-BuOH and a few drops of t-BuOOH) followed by addition of N-methylmorpholine oxide. The mixture will be stirred over night at ambient temperature. After quenching the reaction with a 10 ml solution of sodium hydrogen sulphite (15%) the mixture will be evaporated to dryness. The crude will be purified via HPLC.

### Example 2

### N¹,N³,N⁵-tris(3,5-bis(2,3-dihydroxypropylcarbamoyl)-2,4,6-triiodophenyl)cyclohexane-1,3,5-tricarboxamide

The title compound can also be prepared following the procedure below:

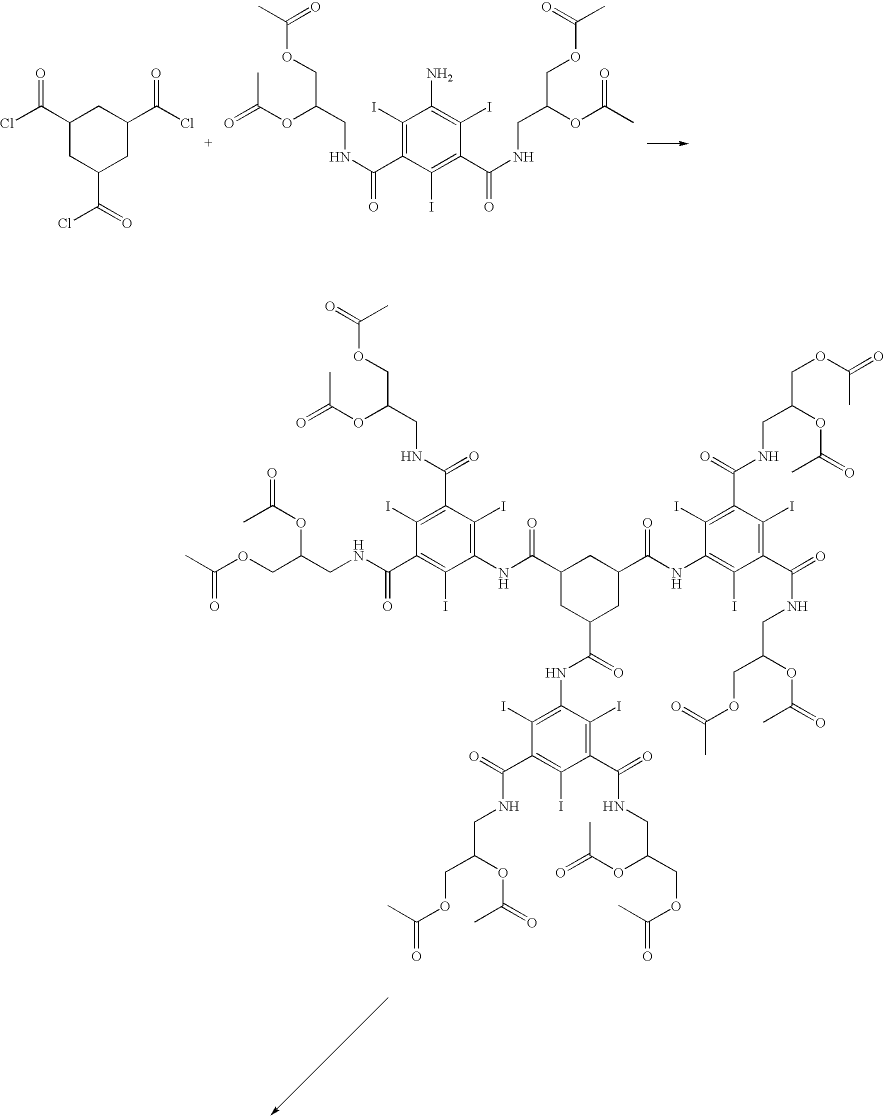

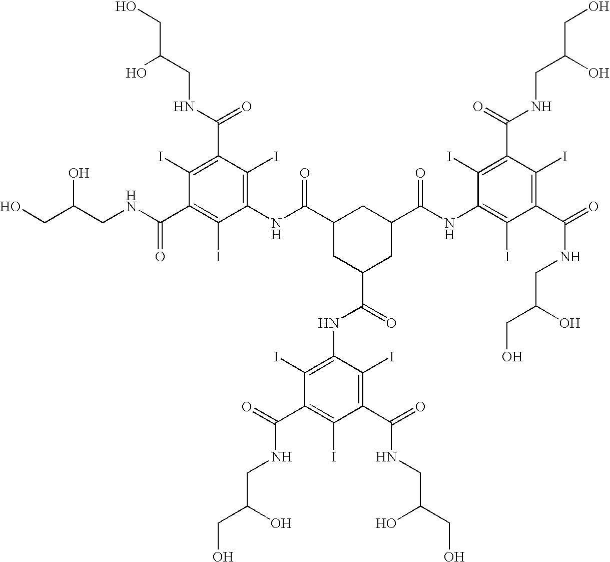

### a) Cyclohexane-1,3,5-tricarboxylic acid tris-{[3,5-bis-(2,3-diacetoxy-propylcarbamoyl)-2,4,6-triiodo-phenyl]-amide}

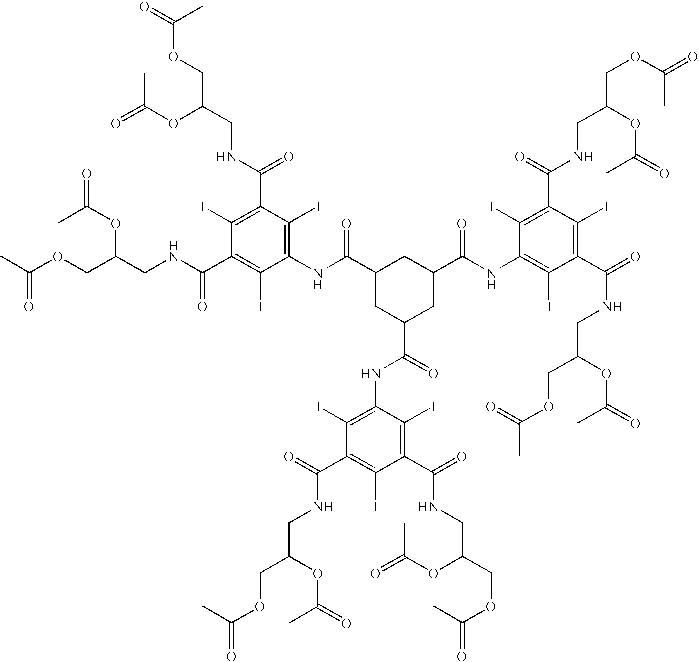

To a cooled solution of acetic acid 1-acetoxymethyl-2-[3-amino-5-(2,3-diacetoxy-propylcarbamoyl)-2,4,6-triiodo-benzoylamino]-ethyl ester (1.33 g, 1.53 mmol) in dimethylacetamide (10 ml) with triethylamine (1.55 mmol) is added cyclohexane-1,3,5-tricarbonyl trichloride (135 mg, 0.5 mmol) in dimethylacetamide (5 ml) dropwise. The mixture is stirred for 3 h and then allowed to ambient temperature overnight. The reaction is poured into ethylacetate (150 ml) and washed with water, dried over MgSO₄ and evaporated. The product is isolated by chromatography on silica gel.

### b) Cyclohexane-1,3,5-tricarboxylic acid tris-{[3,5-bis-(2,3-dihydroxy-propylcarbamoyl)-2,4,6-triiodo-phenyl]-amide}

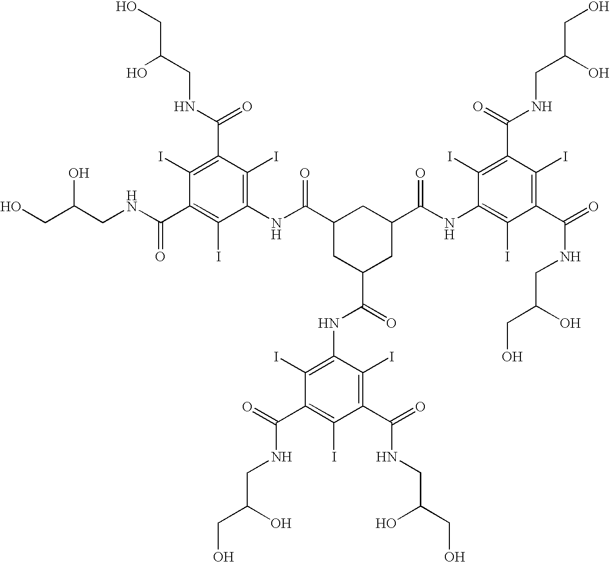

Cyclohexane-1,3,5-tricarboxylic acid tris-{[3,5-bis-(2,3-diacetoxy-propylcarbamoyl)-2,4,6-triiodo-phenyl]-amide}(1.0 g) is dissolved in methanol (10 ml) and concentrated ammonia (3 ml) is added and the solution stirred at ambient temperature overnight. The solution is evaporated to dryness and title product isolated by reverse phase chromatography.

Identified by electrospray mass spec

### Example 3

### 1,3,5-Tris{3[(2,3-dihydroxypropyl)methylcarbamoyl]-5(2,3-dihydroxypropanoylamino)-2,4,6-triiodobenzoylaminomethyl}cyclohexane

The compound is prepared according to the following reaction scheme:

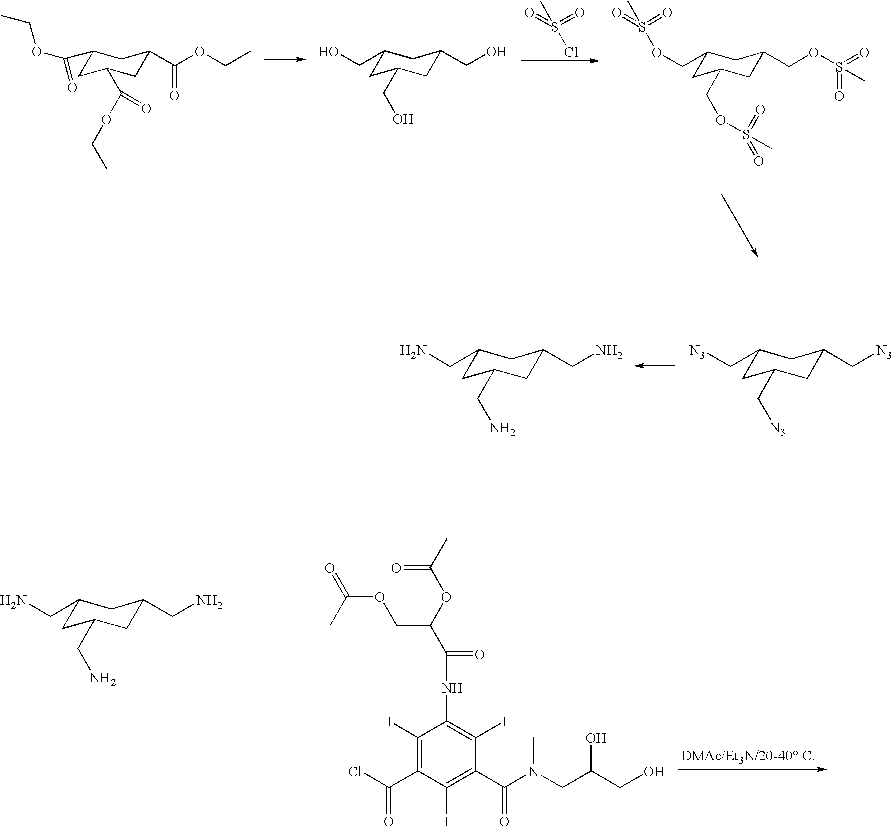

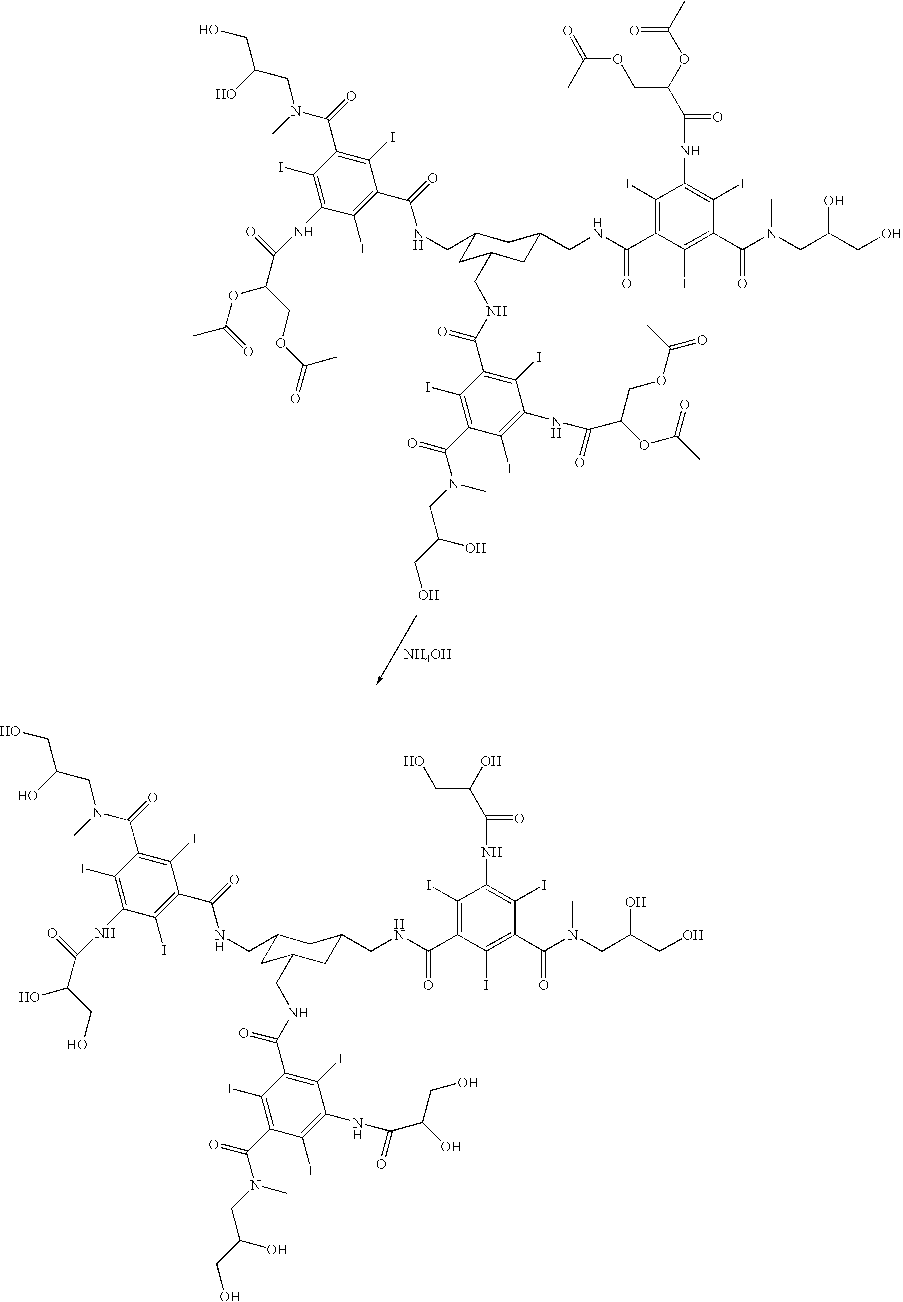

### a) (3,5-Bis-hydroxymethyl-cyclohexyl)-methanol

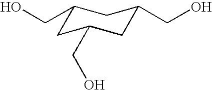

Lithium aluminium hydride (4.4 g, 116 mmol) in THF (100 ml) was treated cautiously with trimethyl cis cis-1,3,5-cyclohexane tricarboxylate (10.0 g, 40 mmol) in THF (50 ml) over the period of ca. 1 h. A strongly exothermic reaction occurred causing the solvent to reflux strongly. The reaction was heated under gentle reflux for 3 days. After the reaction had cooled, acetic acid was added dropwise until H₂ evolution ceased, when acetic anhydride was added. The THF was distilled out and a further portion of acetic anhydride was added to make the mixture mobile. The mixture was then heated at 140° C. for 5 h, during which time the mixture changed from a thick grey paste to a very fine powder. This was then collected by filtration and washed with ethyl acetate. The filtrate was then evaporated to dryness to afford 1,3,5-triacetoxymethylcyclohexane as a yellow liquid in quantitative yield. This was dissolved in 1N HCl (aq)/EtOH and the solution heated under reflux with stirring overnight. The solvent was removed under reduced pressure to give the product triol as a white solid in quantitative yield.

¹H NMR (300 MHz; CD₃OD), 0.56 (3H, q, J_{HH} 12 Hz, CH×3), 1.56 (3H, m, CH×3), 1.87 (3H, m, CH×3), 3.34 (6H, d, J_{HH} 6 Hz, OCH₂).

¹³C NMR (75 MHz; CD₃OD), 32.6 (CH₂), 39.5 (C), 67.3 (OCH₂).

### b) Methanesulfonic acid 3,5-bis-methanesulfonyloxymethyl-cyclohexylmethyl ester

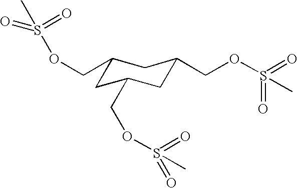

Methane sulfonyl chloride (14 ml, 181.6 mmol) was added slowly to a solution of (3,5-Bis-hydroxymethyl-cyclohexyl)-methanol (9.6 g, 55.1 mmol) and pyridine in anhydrous dichloromethane and stirred at ambient temperature for 18 h. The solvent was removed under vacuum and the residue was washed with water. The residue was extracted with dichloromethane, dried over MgSO4, filtered and evaporated to dryness to give the crude trimesylate (12.5 g, 57%).

¹H NMR (300 MHz; CDCl₃), 0.78-0.95 (3H, m, CH×3), 1.82-1.98 (6H, m, CH₂×3), 3.03 (3H, s, Me), 4.09 (6H, d, J_{HH} 6 Hz, OCH₂×3).

¹³C NMR (75 MHz; CDCl₃), 31.2 (Me), 36.3 (CH₂×3), 37.5 (CH×3), 73.5 (OCH₂).

### c) 1,3,5-Tris-azidomethyl-cyclohexane

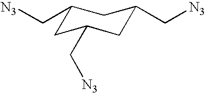

Methanesulfonic acid 3,5-bis-methanesulfonyloxymethyl-cyclohexylmethyl ester (12.0 g, 29 mmol) was dissolved in dry DMF (150 ml) under nitrogen and sodium azide (13.4 g, 206 mmol) was added in portions over five minutes to the stirring solution. The mixture was heated at 50° C. overnight. On cooling, the solution was treated with dilute potassium carbonate solution and extracted three times with petroleum ether:diethyl ether 50:50. The organic extracts were washed with water and dried over sodium sulphate before dilution with ethanol (100 ml) and the solution concentrated in vacuo to ca. 50 ml to remove most of the petroleum ether. CARE! DO NOT REMOVE ALL THE SOLVENT AS THE AZIDE IS POTENTIALLY EXPLOSIVE AND SHOULD BE KEPT IN DILUTE SOLUTION AT ALL TIMES.

A small sample was evaporated to dryness:

¹H NMR (300 MHz; CDCl₃), 0.65-0.81 (3H, m, CH×3), 1.60-1.95 (6H, m, CH×6), 3.21 (6H, d, J_{HH} 6 Hz, NCH₂×3).

¹³C NMR (75 MHz; CDCl₃), 33.2 (CH₂×3), 36.0 (CH₂×3), 57.3 (CH₂N).

### d) C-(3,5-Bis-aminomethyl-cyclohexyl)-methylamine

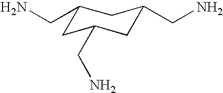

1,3,5-Tris-azidomethyl-cyclohexane (7.5 g, 29.4 mmol) in ethanol (50 ml) was added slowly under a flow of nitrogen to 10% palladium on carbon (˜600 mg). The reaction was subjected to hydrogenation for 40 h at 40° C. The catalyst was filtered off on Celite and evaporated under vacuum to give the product as a waxy solid (5.5 g)

¹³C NMR (75 MHz; CDCl₃), 34.8 (CH×3), 40.3 (CH₂×3), 48.7 (CH₂N).

### e) 1,3,5-Tris{3[(2,3-dihydroxypropyl)methylcarbamoyl]-5(2,3-diacetoxypropanoylamino)-2,4,6-triiodobenzoylaminomethyl}cyclohexane

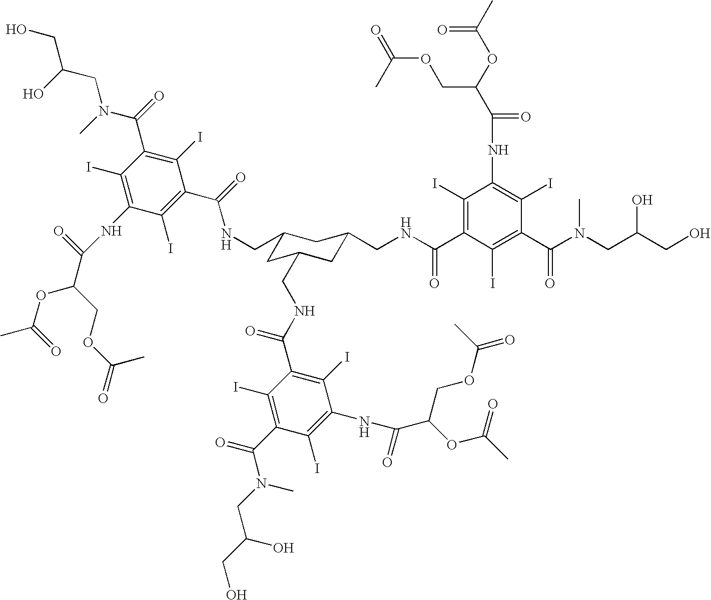

To a solution of C-(3,5-Bis-aminomethyl-cyclohexyl)-methylamine (0.58 g, 3.36 mmol) in dimethylacetamide (20 ml) is added triethylamine (1.99 ml, 14.3 mmol) followed by a solution of acetic acid 2-acetoxy-1-{3-chlorocarbonyl-5-[(2,3-dihydroxy-propyl)-methyl-carbamoyl]-2,4,6-triiodo-phenylcarbamoyl}-ethyl ester (11.24g, 13.4 mmol) in dimethylacetamide (20 ml). The mixture is stirred at 60° C. for 24 h. Excess triethylamine is evaporated at reduced pressure and ethyl acetate (450 ml) is added. The resultant solution is washed with ice-water (4×50 ml), brine, dried (MgSO4) filtered and evaporated to give a brown viscous oil, which is purified by column chromatography on silica gel in 97:3-7:3 ethyl acetate:methanol to give the product as a white solid foam.

### f) 1,3,5-Tris{3[(2,3-dihydroxypropyl)methylcarbamoyl]-5(2,3-dihydroxypropanoylamino)-2,4,6-triiodobenzoylaminomethyl}cyclohexane

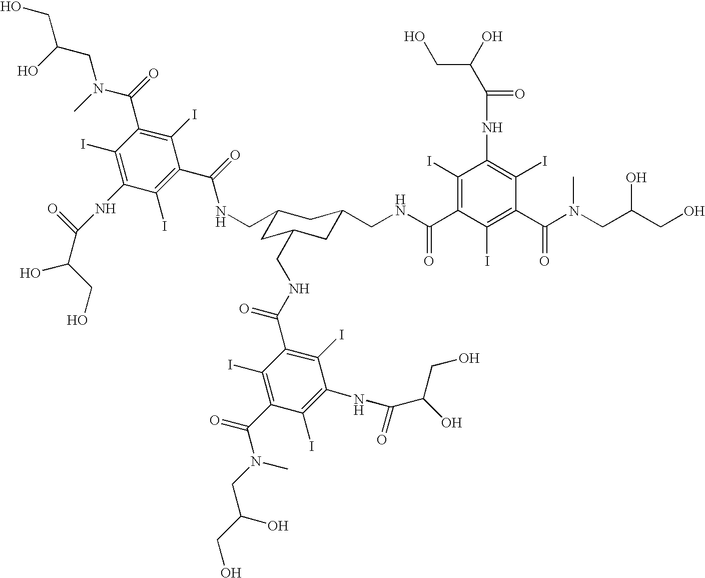

Trimer is dissolved in methanol (20 ml) and concentrated aqueous ammonia (3 ml) added and the solution stirred at ambient temperature for 18 h. Solvent is evaporated and the product is isolated by preparative HPLC using an acetonitrile-water gradient.

The product is identified by its molecular ion in electrospray MS.

### Example 4

### 1,3,5-Tris{3[(2,3-dihydroxypropyl)methylcarbamoyl]-5(2,3,4-trihydroxybutanoylamino)-2,4,6-triiodobenzoylaminomethyl}cyclohexane

### a) 1,3,5-Tris{3[(2,3-dihydroxypropyl)methylcarbamoyl]-5(2,3,4-triacetoxybutanoylamino)-2,4,6-triiodobenzoylaminomethyl}cyclohexane

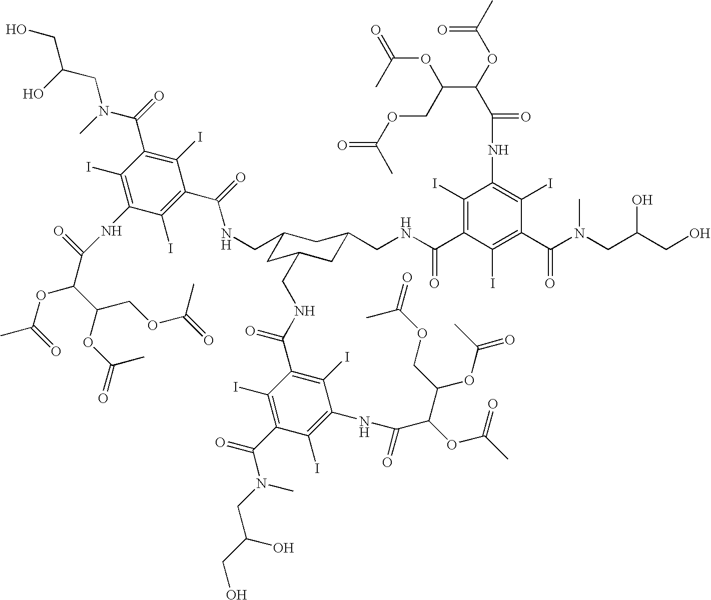

To a solution of C-(3,5-Bis-aminomethyl-cyclohexyl)-methylamine (0.58 g, 3.36 mmol) in dimethylacetamide (20 ml) is added triethylamine (1.99 ml, 14.3 mmol) followed by a solution of acetic acid 2,3-diacetoxy-1-{3-chlorocarbonyl-5-[(2,3-dihydroxy-propyl)-methyl-carbamoyl]-2,4,6-triiodo-phenylcarbamoyl}-propyl ester (12.18 g, 13.4 mmol) in dimethylacetamide (20 ml). The mixture is stirred at 60° C. for 24 h. Excess triethylamine is evaporated at reduced pressure and ethyl acetate (450 ml) is added. The resultant solution is washed with ice-water (4×50 ml), brine, dried (MgSO4) filtered and evaporated to give a brown viscous oil, which is purified by column chromatography on silica gel in 97:3-7:3 ethyl acetate:methanol to give the product as a white solid foam.

### b) 1,3,5-Tris{3[(2,3-dihydroxypropyl)methylcarbamoyl]-5(2,3,4-trihydroxybutanoylamino)-2,4,6-triiodobenzoylaminomethyl}cyclohexane

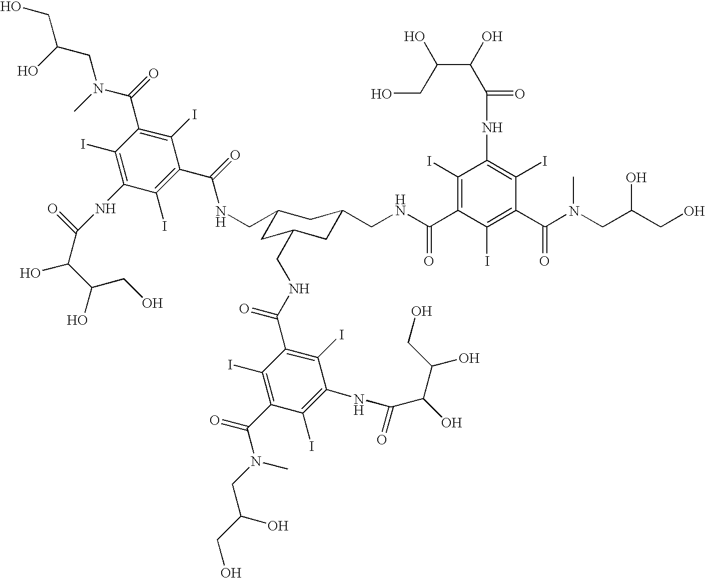

Nona-acetyl trimer is dissolved in methanol (20 ml) and concentrated aqueous ammonia (3 ml) added and the solution stirred at ambient temperature for 18 h. Solvent is evaporated and the product is isolated by preparative HPLC using an acetonitrile-water gradient.

The product is identified by its molecular ion in electrospray MS.

### Example 5

### 1,35-tris-[3-[(2,3-dihydroxy-propyl-methyl-carbamoyl]-5-(2-hydroxy-acetylamino)-2,4,6-triiodo-benzoylamino]-2,4,6-trihydroxy-cyclohexane

### a) 2,4,6-Triamino-cyclohexane-1,3,5-triol

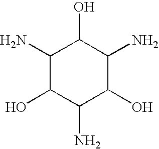

The synthesis of 2,4,6-Triamino-cyclohexane-1,3,5-triol is described in Helv. Chim. Acta.; 1990: 73; 97 and references therein and also illustrated above.

### b) Acetic acid {3-(3,5-bis-{3-(2-acetoxy-acetylamino)-5-[(2,3-dihydroxy-propyl)-methyl-carbamoyl]-2,4,6-triiodo-benzoylamino}-2,4,6-trihydroxycyclohexylcarbamoyl)-5-[(2,3-dihydroxy-propyl)-methyl-carbamoyl]-2,4,6-triiodo-phenylcarbamoyl}-methyl ester

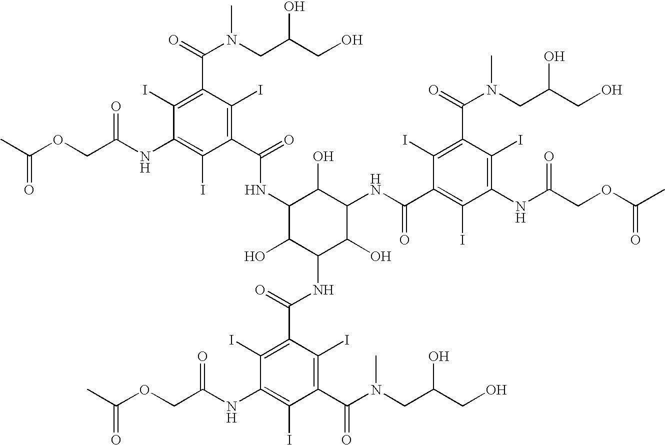

To a solution of acetic acid {3-chlorocarbonyl-5-[(2,3-dihydroxy-propyl)-methyl-carbamoyl]-2,4,6-triiodo-phenylcarbamoyl}-methyl ester in DMAc is added triethylamine (3.3 equivalent) and 2,4,6-Triamino-cyclohexane-1,3,5-triol (0.3 equivalent). The mixture is heated at 40° C. for 18 hours. The reaction mixture is diluted with water and extracted with ethyl acetate. The organics are dried and absorbed onto silics gel. The crude mixture is separated eluting with methanol/DCM (5:95 to 20:80). The desired material is collected, concentrated and used without further purification.

### c) 1,3,5-tris-[3-[(2,3-dihydroxy-propyl)-methyl-carbamoyl]-5-(2-hydroxy-acetylamino)-2,4,6-triiodo-benzoylamino]-2,4,6-trihydroxy-cyclohexane

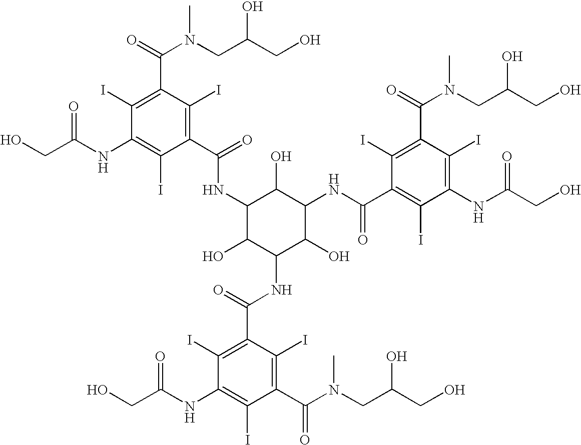

To a solution of acetic acid {3-(3,5-bis-{3-(2-acetoxy-acetylamino)-5-[(2,3-dihydroxy-propyl)-methyl-carbamoyl]-2,4,6-triiodo-benzoylamino}-2,4,6-trihydroxycyclohexylcarbamoyl)-5-[(2,3-dihydroxy-propyl)-methyl-carbamoyl]-2,4,6-triiodo-phenylcarbamoyl}-methyl ester in methanol is added anhydrous ammonia in methanol. The solution is stirred at ambient temperature. The desired material will be isolated using preparative HPLC.

What is claimed is:

1. Compound of formula (I) 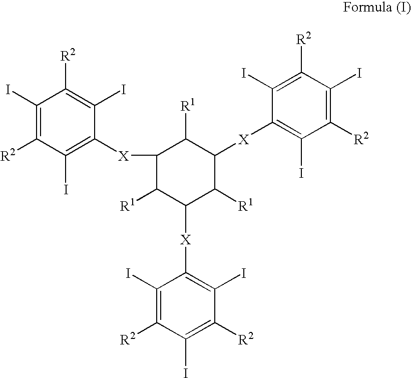
R²
R²
R²′ R²
Formula (I)
and salts or optical active isomers thereof
wherein each R¹ are the same and different and denotes a hydrogen atom or a hydroxyl group;
each R² are the same or different and denote a hydrogen atom or a non-ionic hydrophilic moiety, provided that at least one of the R groups represent a non-ionic hydrophilic moiety;
each X are the same or different and denote a bridging group of the formulas *—(CH₂)ₙ—NR—CO— and *—CO—NR—(CH₂)ₙ— where
R denotes a hydrogen atom or an acyl moiety;
n denotes an integer of 0 to 4; and
* denotes the binding to the central cyclohexane moiety

2. Compound as claimed in claim 1 wherein the hydrophilic moieties R² are the same or different and denotes straight chain or branched chain C₁₋₁₀ alkyl groups, preferably straight chain or branched chain C₁₋₅ alkyl groups optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms.

3. (canceled)

4. Compound as claimed in claim 2 wherein the R² moieties contain 1 to 6, preferably 1 to 3 hydroxy groups.

5. Compound as claimed in claim 4 wherein the R² moieties denote polyhydroxyalkyl, hydroxyalkoxyalkyl and hydroxypolyalkoxyalky groups.

6. Compound as claimed in claim 1 wherein
R² comprises one or more of the following groups:
—CONH—CH₂—CH₂OH
—CONH—CH₂—CHOH—CH₂OH
—CON(CH₃)CH₂—CHOH—CH₂OH
—CONH—CH— (CH₂OH)₂
—CON—(CH₂—CH₂OH)₂
—CON—(CH₂—CHOH—CH₂OH)₂
—CONH₂
—CONHCH₃
—NHCOCH₂OH
—N(COCH₃)H
—N(COCH₃) C₁₋₃ alkyl
—N(COCH₃)— mono, bis or tris-hydroxy C₁₋₄ alkyl
—N(COCH₂OH)— hydrogen, mono, bis or tris-hydroxy C₁₋₄ alkyl
—N(CO—CHOH—CH2OH)— hydrogen, mono, bis or trihydroxylated C₁₋₄ alkyl.
—N(CO—CHOH—CHOH—CH2OH)— hydrogen, mono, bis or trihydroxylated C₁₋₄ alkyl
—CON(CH₂—CHOH—CH₂—OH)(CH₂—CH₂—OH)
—CONH—C(CH₂—OH)₃
—CONH—CH(CH₂—OH)(CHOH—CH₂—OH).

7. Compound as claimed in claim 6 wherein R² are selected from the group of the formulas —CON(CH₃)CH₂—CHOH—CH₂OH, —CONH—CH₂—CHOH—CH₂OH, —CONH—CH— (CH₂OH)₂, —CON—(CH₂—CH₂OH)₂ or —CON—(CH₂—CHOH—CH₂OH)₂, —NHCOCH₂OH and —N(COCH₂OH)— mono, bis or tris-hydroxy C₁₋₄ alkyl.

8. Compound as claimed in claim 7 wherein all R² groups consists of CONH—CH₂—CHOH—CH₂OH.

9. (canceled)

10. Compound as claimed in claim 1 wherein the R¹ groups all denote hydrogen or all denote hydroxyl.

11. Compound as claimed in claim 10 wherein the R¹ groups all denote hydrogen.

12. Compound as claimed in claim 1 wherein all the X groups are the same.

13. Compound as claimed in claim 1 wherein n denotes 0 or 1.

14. Compound as claimed in claim 1 wherein each R denotes hydrogen atoms.

15. Compound as claimed in claim 1 wherein each R denotes residues of aliphatic organic acids.

16. Compound as claimed in claim 15 wherein the aliphatic organic acid denotes a C₂ to C₅ organic acid moiety such as formyl, acetyl, propionyl, butyryl, isobutyryl and valeriyl moieties

17. Compound as claimed in claim 15 wherein all R groups are the same.

18. Compound as claimed in claim 17 wherein all R are the same and denote the acetyl moieties.

19. Compound as claimed in claim 1 wherein
X denotes groups of the formulas *—CH₂—NH—CO— and *—NH—CO— and *—CO—NH.

20. Compound as claimed in claim 1 wherein said compound is
N¹,N³,N⁵-tris(3,5-bis(2,3-dihydroxypropylcarbamoyl)-2,4,6-triiodophenyl)cyclohexane-1,3,5-tricarboxamide;
1,3,5-Tris{3[(2,3-dihydroxypropyl)methylcarbamoyl]-5(2,3-dihydroxypropanoylamino)-2,4,6-triiodobenzoylaminomethyl}cyclohexane;
1,3,5-Tris{3[(2,3-dihydroxypropyl)methylcarbamoyl]-5(2,3,4-trihydroxybutanoylamino)-2,4,6-triiodobenzoylaminomethyl}cyclohexane;
1,3,5-tris-[3-[(2,3-dihydroxy-propyl)-methyl-carbamoyl]-5-(2-hydroxy-acetylamino)-2,4,6-triiodo-benzoylamino]-2,4,6-trihydroxy-cyclohexane.

21. (canceled)

22. A diagnostic composition comprising a compound of formula (I) as defined in claim 1 together with a pharmaceutically acceptable carrier or excipient.

23-25. (canceled)

26. A method of diagnosis comprising administration of compounds of formula (I) as defined claim 1 to the human or animal body, examining the body with a diagnostic device and compiling data from the examination.

27-28. (canceled)
